(19) United States
(12) Patent Application Publication
(10) Pub. No.: US 2021/0052175 A1
(43) Pub. Date: Feb. 25, 2021

(54) SYSTEMS AND METHODS FOR USING CHARACTERISTICS OF PHOTOPLETHYSMOGRAPHY (PPG) DATA TO DETECT CARDIAC CONDITIONS

(71) Applicant: Verily Life Sciences LLC, South San Francisco, CA (US)

(72) Inventors: Philip Stephens, Niwot, CO (US); Michael McConnell, Los Altos Hills, CA (US); Allen Keel, San Jose, CA (US)

(21) Appl. No.: 16/550,087

(22) Filed: Aug. 23, 2019

(51) Int. Cl. A61B 5/024; A61B 5/00

(52) U.S. Cl. CPC A61B 5/02416; A61B 5/02438; A61B 5/721; A61B 5/046; A61B 5/681; A61B 2562/0219; A61B 5/7267

(57) ABSTRACT

In some embodiments, features are extracted from a waveform generated by a photoplethysmograph (PPG) sensor of a wearable device. These features are used to train and use classifier models to detect likely instances of cardiovascular conditions that affect blood flow during a cardiac cycle, including but not limited to atrial fibrillation. In some embodiments, the features are extracted based on the shape of the waveform, including one or more of an amplitude, an upslope rate, a downslope rate, and differentials thereof over time. In some embodiments, data from an inertial measurement unit (IMU) is used to filter and/or compensate for motion artifacts in the PPG data. The use of data generated by PPG sensors allows long-term, non-invasive monitoring for cardiovascular conditions without requiring further actions to be taken by the user to obtain data using other means.

### TECHNICAL FIELD

This disclosure relates generally to cardiac monitoring.

### BACKGROUND

A variety of cardiovascular parameters or other information about the health state of a person can be detected by a wearable device that is mounted to the person's body. Such a wearable device can operate one or more sensors to continuously detect information about the person's body. Such continuous monitoring can provide an increased amount of data to improve the determination of information about the person's health state, e.g., by increasing a confidence level of a health state determination. Additionally, by continuously monitoring the person across extended periods of time and/or across a variety of activities of the person, rare physiological events can be detected. Such rare events could be indicative of a serious health condition, e.g., atrial fibrillation.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a system for monitoring a cardiac condition of a subject is provided. The system comprises a wearable device that includes a photoplethysmograph (PPG) sensor; and a computing system. The computing system is communicatively coupled to the wearable device and that includes logic that, in response to execution by the computing system, causes the computing system to perform actions comprising: receiving PPG waveform data from the wearable device; and providing at least information based on the PPG waveform data to a classifier model to label the PPG waveform data as either including the cardiac condition, not including the cardiac condition, or being indeterminate.

In some embodiments, a non-transitory computer-readable medium is provided. The medium has computer-executable instructions stored thereon that, in response to execution by one or more processors of a computing system, cause the computing system to perform actions for monitoring a subject for a cardiac condition. The computing system obtains photoplethysmograph (PPG) waveform data collected by a wearable device. The computing system generates features based on the PPG waveform data. The computing system provides the features to a classifier model to label the PPG waveform data as either including the cardiac condition, not including the cardiac condition, or being indeterminate.

In some embodiments, a non-transitory computer-readable medium is provided. The medium has computer-executable instructions stored thereon that, in response to execution by one or more processors of a computing system, cause the computing system to perform actions for training a classifier model to detect a cardiac condition. The computing system receives photoplethysmograph (PPG) waveform data collected from subjects of a first population that are known to exhibit the cardiac condition and from subjects of a second population that are known to not exhibit the cardiac condition. The computing system generates features based on the PPG waveform data to create a set of training data for the first population and a set of training data for the second population. The computing system trains a classifier model to label PPG waveform data as either exhibiting the cardiac condition, not exhibiting the cardiac condition, or being indeterminate using the set of training data for the first population and the set of training data for the second population.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A illustrates a non-limiting example embodiment of a wearable device according to various aspects of the present disclosure, mounted to a wrist of an arm of a wearer;

FIG. 1B illustrates the wearable device in detail;

FIG. 2 illustrates a non-limiting example embodiment of a wearable device according to various aspects of the present disclosure;

FIG. 3 is a simplified schematic drawing illustrating a non-limiting example embodiment of a system including one or more wearable devices according to various aspects of the present disclosure;

FIG. 4 is a simplified block diagram illustrating some components included in a non-limiting example embodiment of a device according to various aspects of the present disclosure;

FIG. 5 is a simplified block diagram illustrating some components included in a non-limiting example embodiment of an analysis device according to various aspects of the present disclosure;

FIGS. 6A-6B are a flowchart that illustrates a non-limiting example embodiment of a method of monitoring cardiac activity to detect one or more conditions according to various aspects of the present disclosure;

FIGS. 7A-7B are a flowchart that illustrates a non-limiting example embodiment of a method of training a classifier model to detect a cardiac condition according to various aspects of the present disclosure;

FIG. 8 is a flowchart that illustrates a non-limiting example embodiment of a procedure for determining features for a segment of photoplethysmograph (PPG) data according to various aspects of the present disclosure; and

FIG. 9 is a chart that illustrates a non-limiting example embodiment of a PPG waveform according to various aspects of the present disclosure.

### DETAILED DESCRIPTION

### Overview

It can be beneficial to continuously monitor a subject for the occurrence of certain health events, e.g., cardiovascular events. For example, it could be beneficial to monitor for ventricular or atrial fibrillation, tachycardia, bradycardia, or other arrhythmic events. Detection of cardiac conditions via continuous monitoring can facilitate prompt emergency medical attention, administration of a drug or other therapy, or improved detection of information about the cardiovascular condition.

In order to detect cardiac conditions such as atrial fibrillation (AF), one or more sensors could be incorporated into a wearable device and worn by a subject. The one or more sensors could be operated to detect pulse rates, time-varying blood volume in subsurface vasculature, electrocardiographic signals, the acceleration or rotation of a body part, ambient light levels, or other signals that could be used to determine whether a cardiovascular event is occurring. The determination of whether a cardiovascular event is occurring could involve use of a classifier model to determine whether one or more sensor signals are indicative of the cardiovascular event.

One main issue with regard to monitoring for cardiovascular events or conditions using a wearable device relates to managing a balance between battery life and accuracy. Some existing wearable devices may pair a single-lead ECG with a wearable device that includes a photoplethysmograph (PPG) sensor. However, battery life on such devices is exceedingly low, particularly while monitoring for cardiovascular conditions. This causes such devices to only perform checks for cardiovascular conditions at long intervals in order to conserve battery, and the time needed to recharge causes gaps to appear in the monitoring data. Other devices include only a PPG sensor and have better battery life. However, such devices typically only use the PPG sensor to determine heart rate or inter-beat interval (IBI) information, and so are not capable of generating clinically useful information for detecting many cardiovascular conditions. Implantable loop recorders may be able to provide continuous ECG readings for up to three years, but are expensive and invasive. Likewise, mobile cardiac telemetry (ECG) patches are also expensive, and are not generally suited to long-term monitoring.

Having access to long-term collections of data is important in accurately detecting cardiac conditions, and using a low-power, lower-quality sensor (such as a PPG sensor) is a technique that can be used to continuously monitor a subject. However, even if heart rate or IBI data collected via photoplethysmography is used to determine if there is a possibility that a cardiac condition is present, electrocardiogram (ECG) waveforms are usually collected in order to confirm a specific condition. What is desired are techniques for using the PPG sensor information itself to detect and quantify cardiac conditions, such that further interaction from a user to collect ECG waveforms is unnecessary for diagnosis.

It has been discovered that certain cardiac conditions, such as atrial fibrillation, cause changes in peripheral blood flow during the cardiac cycle. In particular, under atrial fibrillation, the variation in inter-beat interval leads to longer cardiac filling time for some beats, which will result in greater stroke volume and contractility for those beats, which would then be reflected in higher pulse amplitude and upslope rate. This volumetric information is encoded in the raw PPG waveform, but not in the traditional rhythm-based features extracted from PPG sensors, such as inter-beat interval, and is not present in an ECG waveform.

In some embodiments of the present disclosure, classifier models are trained to distinguish between healthy blood flow patterns and blood flow patterns that exhibit changes caused by cardiac conditions. Accordingly, in some embodiments of the present disclosure, cardiac conditions can be reliably detected using PPG data that is not enhanced with data from any other source.

### Training and Using Classifiers to Detect Cardiac Conditions

In order to distinguish healthy blood flow patterns from blood flow patterns that exhibit changes caused by cardiac conditions, a classifier model can be generated based on extensive data from one or more devices (e.g., from devices collecting data from one or more wearers), data from clinical data acquisition systems, or other data sources. Further, the classifier model could be updated over time, e.g., based on signals detected by one or more wearable devices in the future.

Accessing such extensive sources of data (e.g., from clinical systems, from many other wearable devices) and using such data to generate a classifier model could require an amount of data storage, an amount of processor power, access to remote databases, or other factors that are difficult or impractical to provide in a wearable device itself. Instead, a wearable device could upload sensor data to a cloud computing service, a computer at a physician's office or hospital, or some other analysis device. The analysis device could then use the sensor data from the wearable device, along with additional data (e.g., data from other wearable devices, data from clinical data acquisition systems, data from previous clinical studies or other sources of population data), to generate a classifier model.

In some embodiments, the analysis device may then receive PPG data from wearable devices, and use one or more classifier models to check the PPG data for various cardiac conditions. In some embodiments, the analysis device could transmit the determined classifier model to the wearable device, and the wearable device could use the classifier model to determine whether sensor signals generated by the wearable device are indicative of a cardiovascular condition. The wearable device could also receive updated classifier models from the analysis device, e.g., classifier models that have been updated based on additional sensor data received from the wearable device, from other wearable devices, or from some other source. In some embodiments, a classifier model may be placed on the wearable device during manufacture or configuration, and may not be updated further.

A wearable device, e.g., a wrist-mountable device, could include one or more sensors configured to detect physical variables (e.g., to illuminate a portion of subsurface vasculature and detect a volume of blood in the portion of subsurface vasculature based on a detected intensity of responsively emitted light) that are related to the occurrence or non-occurrence of a cardiovascular event of interest. Such cardiovascular events could include atrial or ventricular fibrillation, tachycardia, bradycardia, or other arrhythmias, acute hypertension or hypotension, incidents of postural orthostatic tachycardia syndrome, incidents of clinical or subclinical cardiac arrest or ischemia, extra heart beats, or other events of interest. It could be beneficial to use such a wearable device to continuously monitor a wearer in order to detect the occurrence of such events. Such detection could permit more prompt delivery of medical care, timed application of a drug or other medical treatment, detection of additional information about rare events, or some other beneficial applications.

A variety of physiological or other signals related to a cardiovascular event or to some physiological event or process of interest could be detected and used to determine whether such an event is occurring as described herein. In some examples, such signals could be directly related to the event or process of interest, e.g., a photoplethysmographic signal. Additionally or alternatively, the signals could be related to factors surrounding the process or event of interest, or could provide a context that could facilitate detection of an event of interest. For example, an ambient light level, an orientation, acceleration, or rotation of a body segment over time, a galvanic skin resistance, or some other signals could be used to determine an activity of a wearer, e.g., to determine whether the wearer is resting, walking, sleeping, eating, exercising, or engaging in some other activity. Such a determined activity could then be used to determine whether a sensor signal is indicative of a cardiovascular condition.

For example, a particular detected pulse rate, pulse rate variability, or other signal of interest could be within a range of expected values if a wearer is determined to be exercising. However, if the particular detected pulse rate, pulse rate variability, or other signal of interest is detected while the wearer is sleeping, resting, or otherwise engaged in a non-strenuous activity, it could be indicative that the wearer is experiencing tachycardia, myocardial infarction, or some other cardiovascular event. In such an example, the wearer could be prompted to seek medical attention, to take a drug, to facilitate detection of electrocardiographic or other signals of interest related to the event, or to take some other action.

When a device has determined (using a classifier model or other algorithm) that the output of one or more sensors is indicative of a cardiovascular event, the device may responsively perform some additional operations. For example, the device could prompt a user to seek medical attention, prompt the user to perform a diagnostic task (e.g., to interact with a user interface of the device, to perform a sit-and-stand task), prompt the user to interact with the device in some way (e.g., to use the device to communicate with a health care provider), or perform some other operations.

The device could prompt a user to interact with a sensor of the device in order to permit the detection of a signal of interest and/or to improve a noise level, accuracy, or other quality of a detected signal of interest. For example, the user could be prompted to apply pressure to the device to improve a level of coupling between a sensor and skin of the wearer, to align a sensor with a part of the wearer's body, to control a degree of perfusion of subsurface vasculature, to control a pressure applied to subsurface vasculature (e.g., to detect a blood pressure), or to perform some other action.

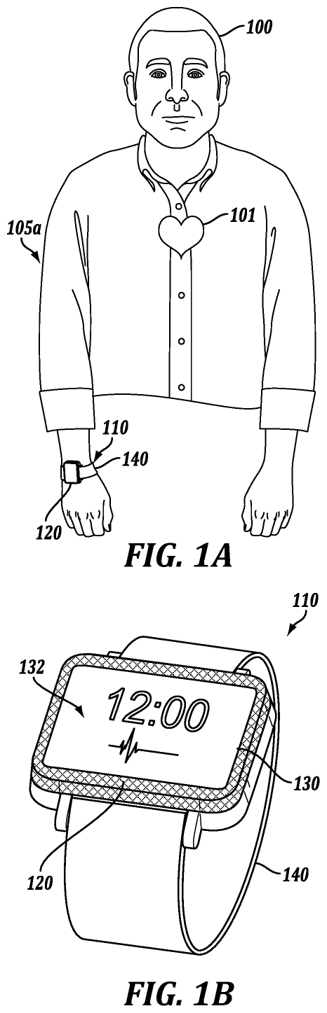

FIG. 1A illustrates a non-limiting example embodiment of a wearable device 110 according to various aspects of the present disclosure, mounted to a wrist of an arm 105a of a wearer 100. The wearable device 110 includes a housing 120 mounted to the wrist of the arm 105a by a mount 140 (e.g., a strap or band). FIG. 1B illustrates the wearable device 110 in detail. The housing 120 has an outside surface that is away from the first external surface of the body and an inside surface (not shown) that is toward and/or in contact with the first external surface of the body when the housing 120 is positioned on the first external surface of the body. A user interface 132 is disposed on the outside surface of the housing 120. Other configurations of a wearable device as described herein are anticipated.

A classifier model can be used to determine whether one or more sensor signals are indicative of the cardiovascular condition of interest. A classifier model can apply thresholds, filters, pattern-matching templates, linear or nonlinear kernels or transforms, finite state machines, statistical inference, or other algorithms or techniques, individually or in combination, to determine whether a cardiovascular condition is occurring, or is likely occurring, based on one or more sensor signals detected from a wearer's body and/or from an environment of the wearer. Such a determination could include generating a binary output related to the presence of the cardiovascular event and/or generating a likelihood or other value corresponding to the probability, in view of one or more sensor signals, that the cardiovascular condition is occurring. In some embodiments, multiple determinations for segments of data over an extended time period could be used to quantify an overall burden of a cardiovascular condition.

Using a classifier model to determine whether a signal is indicative of a cardiovascular condition could include transforming the signal in some way and/or determining some representative information about the signal. For example, the signal could be bandpass filtered, lowpass filtered, highpass filtered, applied to a moving-average filter, convolved with a matching filter, thresholded, applied to a polynomial, or filtered or modified in some other way. The signal could be resampled or separated into discrete overlapping or non-overlapping portions (e.g., into individual portions corresponding to respective different heart beats). The signal could be transformed, e.g., using the Fourier transform, the Laplace transform, a wavelet transform, or some other transformation to generate frequency components or other transformed information about the signal. Pulses, peaks, heart beats, electrocardiographic complexes, or other features could be detected within the signal and used to generate information about the signals, e.g., to determine the presence or timing of the features, to determine a shape of the features, to determine a rate of occurrence of a feature and/or to determine a variability of such a rate over time, to determine a deviation of the shape of the features from a template, or to determine some other information about features in the signal. Specific features that could be generated to characterize PPG data are described in detail below.

Using a classifier model to determine whether a signal is indicative of a cardiovascular event could include using a linear or nonlinear kernel or matrix to generate an output based on a vector of inputs related to the sensor signal (e.g., based on a vector of samples of the signal, based on a vector of frequency components of the signal, based on a vector of properties of pulses or other features within the signal). For example, linear or nonlinear principal components analysis, independent component analysis, a support vector machine, or some other algorithm could be used to determine one or more output values based on a signal. Such output values could then be thresholded, applied to a sigmoid or other nonlinear function, or used in some other manner according to the classifier model to determine whether the sensor signal is indicative of the cardiovascular condition of interest.

Using a classifier model to determine whether a signal is indicative of a cardiovascular event could include using pattern matching to determine whether the signal, or some information determined from the signal, is indicative of the cardiovascular event. This could include determining a degree similarity between the sensor signal, frequency components of the sensor signal, an output of another element of the classifier (e.g., the output of a principal components analysis), or some other information related to the signal and a template pattern. Such a degree of similarity could be used to determine that the cardiovascular condition is occurring and/or to determine the likelihood that the cardiovascular condition is occurring. Additionally or alternatively, a template pattern could be convolved with the signal and/or with an output determined from the signal and the result of the convolution could be used to determine whether the signal is indicative of the cardiovascular condition (e.g., if the output of the convolution exceeds a threshold value for more than a threshold duration). Additionally or alternatively, such pattern matching could be used to detect features within the sensor signal and/or to determine information about such features (e.g., to determine a character of the deviation of such features from a template pattern).

Using a classifier model to determine whether a signal is indicative of a cardiovascular condition could include using multiple signals to detect the occurrence or likelihood of occurrence of the cardiovascular condition. This could include applying two or more signals to respective elements of the classifier model (e.g., to respective principal components analyses) and combining the outputs of the elements (e.g., by element-wise multiplication of outputs of respective different principal components analyses) or combining the information from two different signals in some other way (e.g., by convolving the signals together). Additionally or alternatively, one or more of the signals could be used to set a filter parameter, finite state machine state, operating mode, internal variable, or other property of operation of the classifier model which could then be used to determine whether some additional or alternative one or more of the signals is indicative of the cardiovascular condition. For example, one or more signals (e.g., accelerometer signals, gyroscope signals, ambient light signals) could be used to determine an activity of the wearer (e.g., sleeping, exercising, walking, sitting, eating). The classifier model could then determine, based on the detected activity, whether some other sensor signal is indicative of the cardiovascular condition.

In some embodiments, the classifier model may include a machine learning model that accepts features based on the signals as input and outputs labels for the signals. The machine learning model may include any suitable type of machine learning model for generating such labels, including but not limited to a support vector machine, a neural network, a decision tree, a naïve Bayes classifier, and a nearest neighbor method. Such a machine learning model may be trained using any suitable supervised learning technique, including but not limited to gradient descent. In some embodiments, specific features may be extracted from the signals and provided to the classifier model in order to improve detection of specific cardiac conditions. Some example features for improving detection of atrial fibrillation (and/or other cardiac conditions that affect peripheral blood flow) are described in further detail below.

It can be beneficial to reduce the false-positive detection of such cardiovascular conditions (e.g., to improve wearer compliance with performing diagnostic tasks or providing improved sensor signals) and to reduce the false-negative non-detection of such conditions (e.g., to prevent loss of data about an event of interest). To improve the detection of cardiovascular conditions, based on continuous monitoring of one or more related physiological properties or processes related thereto, the classifier model could be determined and/or updated based on sensor data from one or more wearable devices or from other sources of information (e.g., clinical data acquisition systems). The classifier model for a particular device of a particular wearer could be determined based on days, months, or years of sensor data detected by one or more wearable device(s) of the particular wearer. The classifier model for a particular device of a particular wearer could also be determined based on data from other persons (e.g., data from wearable devices of other wearers). For example, the classifier model could be determined based on information from clinical data acquisition systems (e.g., Holter monitors) that are applied and operated by clinicians and/or from a population of wearers using respective wearable devices as described herein.

The classifier model could be updated over time, based on additional sensor signals received from one or more wearable devices or from some other systems (e.g., clinical data acquisition systems). Such updates could occur semi-continuously or according to some other timing. The classifier model could be updated based on signals used to confirm that a particular sensor signal was indicative of a cardiovascular condition. For example, a first instance of a cardiovascular signal could be used to determine that a first sensor signal is indicative of a cardiovascular condition. In response to that determination, a wearable device could provide a prompt to a wearer such that an electrocardiographic waveform or other signal related to the putative based cardiovascular condition is detected. The detected electrocardiographic waveform or other related signal could then be used to determine whether the first sensor signal was actually indicative of the cardiovascular condition and that determination could be used to update the classifier model.

Using such extensive and/or various data to generate and/or update a classifier model could require an amount of data storage, an amount of processor power, access to remote databases, or other factors such that it is not feasible for the classifier model to be generated and/or updated by a wearable device. Rather, such a wearable device could upload sensor data to a cloud computing service, a computer at a physician's office or hospital, or some other analysis device. The analysis device could then use the sensor data from the wearable device, along with additional data (e.g., data from other wearable devices, data from clinical data acquisition systems, data from previous clinical studies or other sources of population data, features derived from the sensor data), to generate a classifier model. The analysis device could then transmit the determined classifier model to the wearable device, and the wearable device could use the classifier model to determine whether future sensor signals generated by the wearable device are indicative of a cardiovascular condition. The wearable device could also receive updated classifier models from the analysis device, e.g., classifier models that have been updated based on additional sensor data received from the wearable device, from other wearable devices, or from some other source. Alternatively, analyzing the waveforms generated by the sensors of the wearable device may also require a large amount of data storage and/or processor power. In such cases, the wearable device may transmit the raw waveforms to the analysis device for generation of features to be provided as input to the classifier models.

### Example Devices

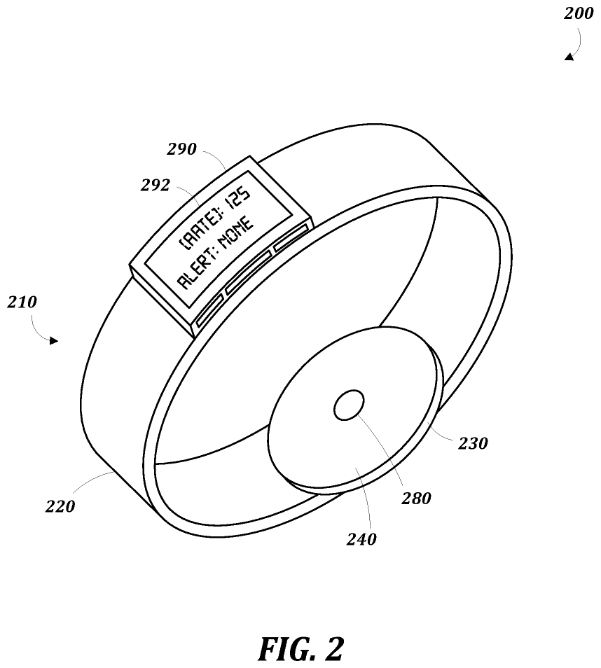

One or more devices or systems could be configured to detect and record a signal, and to provide the records of the signal to an analysis device. The analysis device may then apply a classifier model to the detected signal to determine whether a cardiovascular condition is likely occurring. The analysis device may also train such a classifier model. FIG. 2 illustrates a non-limiting example embodiment of a wearable device 200 according to various aspects of the present disclosure that can record such signals and transmit the signals to an analysis device, or perform other operations described herein.

The term “wearable device,” as used in this disclosure, refers to any device that is capable of being worn at, on or in proximity to a body surface, such as a wrist, ankle, waist, chest, or other body part. In order to take in vivo measurements in a non-invasive manner from outside of the body, the wearable device may be positioned on a portion of the body where a signal related to a cardiovascular condition may be detected (e.g., proximate a portion of subsurface vasculature or some other tissue containing pulsatile blood flow), the qualification of which will depend on the type of detection system used. The device may be placed in close proximity to skin or tissue, but need not be touching or in intimate contact therewith. A mount 210, such as a belt, wristband, ankle band, clip, etc. can be provided to mount the device at, on or in proximity to the body surface. The mount 210 may prevent the wearable device from moving relative to the body to reduce measurement error and noise. In one example, shown in FIG. 2, the mount 210, may take the form of a strap or band 220 that can be worn around a part of the body. Further, the mount 210 may be an adhesive substrate for adhering the wearable device 200 to the body of a wearer.

A measurement platform 230 is disposed on the mount 210 such that it can be positioned on the body where subsurface vasculature is easily observable or where some other signal of interest may be detected. An inner face 240 of the measurement platform is intended to be mounted facing to the body surface. The measurement platform 230 may house first sensor 280, which may be configured to detect one or more signals related to occurrence of a cardiovascular condition of interest (e.g., atrial or ventricular tachycardia, bradycardia, or fibrillation, acute hypotension, acute hypertension, or some other event related to the cardiovascular system of a person). For example, the first sensor 280 may include an optical sensor that is configured to detect a degree of absorption of light at one or more wavelengths by blood in a portion of subsurface vasculature over time (e.g., by illuminating the portion of subsurface vasculature and detecting an intensity or other properties of light responsively reflected by, scattered by, or otherwise emitted from the portion of subsurface vasculature). In another example, the first sensor 280 may include an accelerometer, a pressure sensor, or some other sensor configured to detect a blood pressure in the portion of subsurface vasculature, to detect a displacement of the skin surface related to changes in the volume or pressure of blood in the portion of subsurface vasculature or to motion of a body part (e.g., during exercise, walking, sleeping, or other activities), or to detect some other physical variable related to occurrence or non-occurrence of a cardiovascular event.

The measurement platform 230 may include multiple such sensors, and the signals detected using the sensor(s) could be substantially continuously related to a cardiovascular condition or signal related thereto (e.g., related to a cardiovascular pulse or other physiological signal or process related to the cardiovascular pulse) or could be intermittently related to the cardiovascular condition or signal related thereto (e.g., when the absolute or relative (to a target tissue, e.g., skin surface, portion of subsurface vasculature) motion of the sensor is minimal, when the sensor is in consistent contact with skin or with some other tissue). The wearable device 200 may also include a user interface 290 via which the wearer of the device may receive one or more recommendations, prompts, alerts, or other indications generated either from an analysis device or other computing device, or from a processor within the device. The indications could be any indication that can be noticed by the person wearing the wearable device. For example, the indication could include a visual component (e.g., textual or graphical information on a display), an auditory component (e.g., an alarm sound), and/or tactile component (e.g., a vibration). Further, the user interface 290 may include a display 292 where a visual indication of the alert, prompt, or recommendation may be displayed. The display 292 may further be configured to provide an indication of the measured physiological parameters, for instance, a determined cardiovascular pulse rate, or an indication of a detected cardiovascular condition.

A wearable device as described herein (e.g., wearable device 210) could be modular. That is, one or more components of such a wearable device could be replaceable, extensible, and/or otherwise reconfigurable to add and/or remove capabilities of the wearable device. For example, a wearable device could include a housing containing a battery, a communications interface, a touchscreen user interface, and general-purpose electronics to enable a variety of applications of a wearable device. The wearable device could further include a modular mount configured to mount the housing to an external body surface and to enable some applications of the wearable device, e.g., by including one or more sensors. For example, a first modular mount could be configured to mount the housing around a wrist of a wearer and to enable extraction of an electrocardiographic waveform from voltage fluctuations between the arms of a wearer by providing a second electrical contact on an outside surface of the mount (e.g., an outer surface of a frame encircling the housing) to complement a first electrical contact provided by the housing on an inside surface of the housing. A second modular mount could be configured to mount the housing around the chest of a wearer and to enable detection of breathing patterns of the wearer by providing a strain sensor in a band of the mount that encircles the chest of the wearer. Elements of such a modular device could be electrically connected via, e.g., spring-loaded contacts.

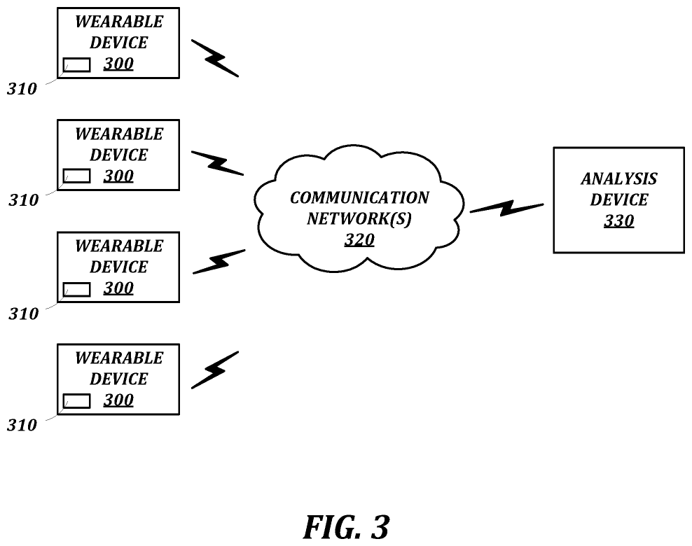

FIG. 3 is a simplified schematic drawing illustrating a non-limiting example embodiment of a system including one or more wearable devices 300 according to various aspects of the present disclosure. The one or more wearable devices 300 may be configured to transmit data via a communication interface 310 over one or more communication networks 320 to an analysis device 330. In one embodiment, the communication interface 310 includes a wireless transceiver for sending and receiving communications to and from the analysis device 330. In further embodiments, the communication interface 310 may include any means for the transfer of data, including both wired and wireless communications. For example, the communication interface may include a universal serial bus (USB) interface or a secure digital (SD) card interface. Communication networks 320 may be any one of may be one of: a plain old telephone service (POTS) network, a cellular network, a fiber network and a data network. The analysis device 330 may include any type of computing device or cloud computing system. The analysis device 330 may include a computing system that comprises one or more computing devices. Further, communication network 320 may include one or more intermediaries, including, for example wherein the wearable device 300 transmits data to a mobile phone or other personal computing device, which in turn transmits the data to the analysis device 330.

In addition to receiving communications from the wearable device 300, such as collected physiological parameter data and data regarding health state as input by the user, the analysis device may also be configured to gather and/or receive either from the wearable device 300 or from some other source, information regarding a wearer's overall medical history, environmental factors and geographical data. For example, a user account may be established on the analysis device for every wearer that contains the wearer's medical history. Moreover, in some examples, the analysis device 330 may be configured to regularly receive information from sources of environmental data, such as viral illness or food poisoning outbreak data from the Centers for Disease Control (CDC) and weather, pollution and allergen data from the National Weather Service. Further, the analysis device may be configured to receive data regarding a wearer's health state from a hospital or physician. Such information may be used in the analysis device's decision-making process, such as recognizing correlations and in generating clinical protocols.

Additionally, the analysis device may be configured to gather and/or receive the date, time of day and geographical location of each wearer of the device during each measurement period. Such information may be used to detect and monitor spatial and temporal spreading of diseases. As such, the wearable device may be configured to determine and/or provide an indication of its own location. For example, a wearable device may include a GNSS system so that it can include GNSS location information (e.g., GPS coordinates) in a communication to the analysis device. As another example, a wearable device may use a technique that involves triangulation (e.g., between base stations in a cellular network) to determine its location. Other location-determination techniques are also possible.

The analysis device 330 may also be configured to make determinations regarding cardiovascular conditions of a user based on information received from one or more of the wearable devices 300 that are associated with the user. This could include receiving signals detected by multiple sensors of a single wearable device 300 and/or receiving signals from multiple devices 300 and using the received signals to determine some information about cardiovascular conditions of a user, e.g., about the existence, timing, or other properties of one or more such events, predictive properties or features of one or more sensor signals in relation to such events, or some other information. The analysis device 330 could also determine information about the sensor signal(s) that could be used by one or more of the devices 300 to determine, based on such sensor signals, whether a cardiovascular condition is present. For example, the analysis device 330 could determine pattern-matching templates, filter cutoffs, parameters of a predictive algorithm, or some other information related to a classifier model that could be transmitted to one of the devices 300. Such a classifier model could then be used by the device 300 to predict, based on signals generated by one or more sensors of the device 300, to predict whether a cardiovascular condition is present. The analysis device 330 could generate such a classifier model based on information received from the particular device 300 and/or based on information received from one or more populations of devices 300. Further, the analysis device 330 may periodically update such a classifier model (e.g., based on additional sensor signals received from the device 300) and send the updated classifier to the device 300.

The analysis device may also be configured to make determinations regarding drugs or other treatments received by a wearer of one or more of the devices 300 and, at least in part, the cardiovascular condition data, detected electrocardiographic signals, and/or the indicated health state of the user. From this information, the analysis device may be configured to derive an indication of the effectiveness of the drug or treatment. For example, if a wearer is prescribed a drug intended to treat tachycardia, but the analysis device receives data from the wearable device(s) indicating (based on determined pulse rates) that the wearer's heart rate has remained elevated over a certain number of measurement periods, the analysis device may be configured to derive an indication that the drug is not effective for its intended purpose for this wearer.

Further, some embodiments of the system may include privacy controls which may be automatically implemented or controlled by the wearer of the device. For example, where a wearer's collected physiological parameter data and health state data are uploaded to a cloud computing network for trend analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as classifier models or collected signals, collected from his or her device may only be used for generating an individual baseline and recommendations in response to collection and comparison of his or her own data and may not be used in generating a population baseline or for use in population correlation studies.

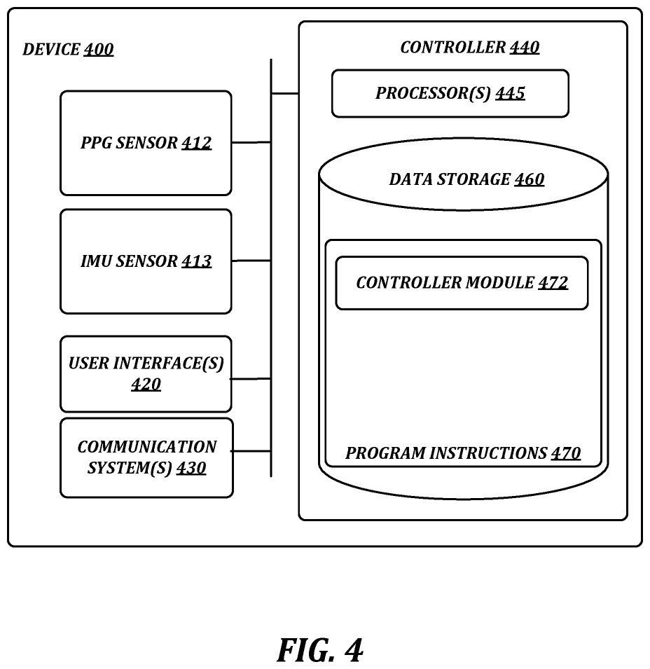

FIG. 4 is a simplified block diagram illustrating some components included in a non-limiting example embodiment of a device 400 according to various aspects of the present disclosure. Device 400 may take the form of or be similar to the devices 110, 200 shown in FIGS. 1A, 1B and 2. That is, device 400 may take the form of a wrist-mountable or otherwise body-mountable device. Device 400 may also take other forms, e.g., could take the form of a device configured to be maintained in proximity to an environment of interest (e.g., a body part) by a user or operator of the device 400 or by a frame or other supporting structure. Device 400 could also take the form of a device configured to obtain signals of interest from some other environment, for example, a body of an animal or some other environment containing a parameter or variable that contains an oscillating pattern having a frequency or rate that could be detected according to the methods described herein. Device 400 also could take other forms.

In particular, FIG. 4 shows an example of a device 400 having a photoplethysmograph (PPG) sensor 412, a user interface 420, communication system(s) 430 for transmitting data to a remote system, and a controller 440. In some embodiments, the device 400 may also include one or more inertial measurement unit (IMU) sensors 413 configured to generate data based on movement of the device 400. The components of the device 400 may be disposed on a mount or on some other structure for mounting the device to enable stable detection of one or more signals related to a cardiovascular condition (e.g., related to a cardiovascular pulse rate) or other process of interest, for example, around a wrist of a wearer such that signals related to a portion of subsurface vasculature or other target tissue are detectable.

Controller 440 may be provided, as illustrated, as a computing device that includes one or more processors 445 that are configured to execute computer-readable program instructions 470 that are stored in the computer readable data storage 460 and that are executable to provide the functionality of a device 400 described herein. In some embodiments, the controller 440 may be provided as an ASIC, FPGA, or other device that incorporates some or all of the logic embodied by the computer-readable program instructions 470 into circuitry.

The computer readable data storage 460 may include or take the form of one or more non-transitory, computer-readable storage media that can be read or accessed by at least one processor 445. The one or more computer-readable storage media can include volatile and/or non-volatile storage components, such as optical, magnetic, organic or other memory or disc storage, which can be integrated in whole or in part with at least one of the one or more processors 445. In some embodiments, the computer-readable data storage 460 can be implemented using a single physical device (e.g., one optical, magnetic, organic or other memory or disc storage unit), while in other embodiments, the computer readable data storage 460 can be implemented using two or more physical devices.

The program instructions 470 stored on the computer readable data storage 460 may include instructions to perform any of the methods described herein. For instance, in the illustrated embodiment, program instructions 470 include a controller module 472.

The controller module 472 can include instructions for operating the PPG sensor 412. For example, the controller module 472 may include instructions for operating a light source and light sensor of the PPG sensor 412 at a plurality of points in time to obtain a respective plurality of samples of a photoplethysmographic signal. The controller module 472 may include instructions for storing records of the plurality of samples in the data storage 460, and to transfer records from the data storage 460 to a analysis device via the communication system(s) 430. In some examples, the controller module 472 may operate an analog-to-digital converter (ADC) to sample one or more signals (e.g., amplifier outputs) generated by the PPG sensor 412 to obtain sets of samples of the signals detected during one or more periods of time.

The controller module 472 can also include instructions for operating a user interface 420. For example, controller module 472 may include instructions for displaying data collected by the controller module 472, for presenting prompts to perform diagnostic tasks or other actions, or for providing some other indications. Further, controller module 472 may include instructions to execute certain functions based on inputs accepted by the user interface 420, such as inputs accepted by one or more buttons or touchscreen displays disposed on the user interface.

Some of the program instructions of the controller module 472 may, in some examples, be stored in a computer-readable medium and executed by a processor located external to the device 400. For example, the device 400 could be configured to operate the PPG sensor 412 (or to otherwise generate or obtain a plurality of samples of a signal related to a cardiovascular event) and then transmit related data to an analysis device, which may include a mobile device, a personal computer, the cloud, or any other system, for further processing (e.g., for the detection of cardiovascular conditions and/or properties or signals related thereto using methods described herein).

User interface 420 could include indicators, displays, buttons, touchscreens, head-mounted displays, and/or other elements configured to present information about the device 400 to a user and/or to allow the user to operate the device 400. Additionally or alternatively, the device 400 could be configured to communicate with another system (e.g., a cellphone, a tablet, a computer, an analysis device) and to present elements of a user interface using the system. The user interface 420 could be disposed proximate to the PPG sensor 412 or other elements of the device 400 or could be disposed away from other elements of the device 400 and could further be in wired or wireless communication with the other elements of the device 400. The user interface 420 could be configured to allow a user to specify some operation, function, or property of operation of the device 400. The user interface 420 could be configured to present a determined pulse rate, cardiovascular condition, or some other health state of a wearer of the device 400, or to present some other information to a user. For example, the user interface 420 could be operated, in response to determining that a cardiovascular condition is present or is likely present, to prompt the wearer to perform some action related to the cardiovascular event (e.g., to perform a diagnostic task, to seek medical attention, to take a drug, to touch an electrical contact of the device 400). Other configurations and methods of operation of a user interface 420 are anticipated.

Communication system(s) 430 may also be operated by instructions within the program instructions 470, such as instructions for sending and/or receiving information via a wired or wireless medium using a transceiver, which may be disposed on or in the device 400. The communication system(s) 430 can optionally include one or more oscillators, mixers, frequency injectors, etc. to modulate and/or demodulate information on a carrier frequency to be transmitted and/or received by an antenna. In some examples, the device 400 is configured to indicate an output from the controller 440 by transmitting an electromagnetic or other wireless signal according to one or more wireless communications standards (e.g., Bluetooth, WiFi, IRdA, ZigBee, WiMAX, LTE) using communication system(s) 430. In some examples, the communication system(s) 430 could include one or more wired communications interfaces and the device 400 could be configured to indicate an output from the controller 440 by operating the one or more wired communications interfaces according to one or more wired communications standards (e.g., USB, FireWire, Ethernet, RS-232).

In some examples, obtained samples of a signal or other physiological property or parameter of interest, determined information about cardiovascular conditions, or other information generated by the device 400 may additionally be input to a cloud network and be made available for download by a user's physician. Analyses may also be performed on the collected data, such as estimates of pulse rate variability, arrhythmia, determinations of post-surgical treatment or rehabilitation regimens, and/or efficacy of drug treatment regimens, in the cloud computing network and be made available for download by physicians or clinicians. Further, collected information from individuals or populations of device users may be used by physicians or clinicians in monitoring efficacy of a surgical intervention or other treatment.

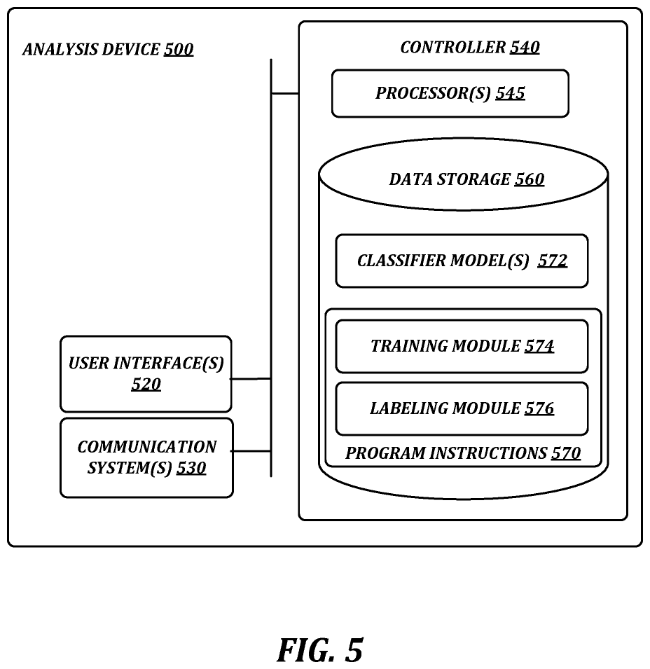

FIG. 5 is a simplified block diagram illustrating some components included in a non-limiting example embodiment of an analysis device according to various aspects of the present disclosure. In some embodiments, analysis device 500 may be a computing device such as a desktop computing device, a laptop computing device, or a rack-mount computing device. In some embodiments, the functionality of analysis device 500 may be provided by more than one computing device working in concert, such as a cloud computing system or other computing system.

In particular, FIG. 5 shows an example of a device 500 having a user interface 520, communication system(s) 530 for receiving data from and transmitting data to a plurality of devices 400, and a controller 540. Controller 540 may be provided, as illustrated, as a computing device that includes one or more processors 545 that are configured to execute computer-readable program instructions 570 that are stored in the computer readable data storage 560 and that are executable to provide the functionality of a device 500 described herein. In some embodiments, the controller 540 may be provided as an ASIC, FPGA, or other device that incorporates some or all of the logic embodied by the computer-readable program instructions 570 into circuitry.

The computer readable data storage 560 may include or take the form of one or more non-transitory, computer-readable storage media that can be read or accessed by at least one processor 545. The one or more computer-readable storage media can include volatile and/or non-volatile storage components, such as optical, magnetic, organic or other memory or disc storage, which can be integrated in whole or in part with at least one of the one or more processors 545. In some embodiments, the computer-readable data storage 560 can be implemented using a single physical device (e.g., one optical, magnetic, organic or other memory or disc storage unit), while in other embodiments, the computer readable data storage 560 can be implemented using two or more physical devices or by a separate computing system accessible by the controller 540 via a wired or wireless network.

The program instructions 570 stored on the computer readable data storage 560 may include instructions to perform any of the methods described herein. For instance, in the illustrated embodiment, program instructions 570 include a training module 574 and a labeling module 576. In some embodiments, the training module 574 may be configured to receive data from a plurality of devices 400 (or other sources) from known populations of subjects, and may train one or more classifier models 572 to detect one or more cardiac conditions. In some embodiments, the labeling module 576 may be configured to receive data from a device 400 from a subject with an unknown health profile, and may use the one or more classifier models 572 to detect one or more cardiac conditions reflected in the data. The labeling module 576 may also be configured to transmit the detected one or more cardiac conditions back to the device 400 in order to prompt the subject to take action. The labeling module 576 may also cause the user interface 520 to present a result of analysis of the data.

The classifier models 572 may include one or more sets of parameters, weights, logic or other information that may be trained by the training module 574 and used by the labeling module 576 for labeling data. The classifier model 572 may include instructions for analyzing data (e.g., signals detected by the PPG sensor 412 and possibly an IMU sensor 413) to determine whether a cardiovascular condition is present (e.g., to determine that a signal received from the sensor is indicative of the cardiovascular condition). In particular, the classifier model 572 may include instructions for determining spectral contents, detecting features (e.g., heart beats), determining pulse rates or pulse timings, applying kernel methods (e.g., principal components analysis or independent components analysis kernels), for generating a priori or a posteriori probabilities, or for performing some other analyses related to determining, based on one or more sensor signals, whether a cardiovascular condition is present. In particular, the classifier model 572 may include algorithmic parameters, PCA or ICA kernels, polynomial coefficients, threshold values, templates for pattern matching, filter coefficients, or other information that could be used to determine whether a particular sensor signal or signals is indicative of a cardiovascular event. In some examples, the classifier model 572 may include instructions for determining a particular activity of the user (e.g., exercising, sleeping, walking, sitting, eating) and determining whether a sensor signal is indicative of the cardiovascular condition could include making such a determination based on the determined activity of the user (e.g., selecting a threshold value based on the determined activity, and applying the selected threshold value to a determined pulse rate to determine whether tachycardia or some other cardiovascular arrhythmic event is occurring).

Communication system(s) 530 may also be operated by instructions within the program instructions 570, such as instructions for sending and/or receiving information via a wired or wireless medium using a transceiver, which may be disposed on or in the device 500. The communication system(s) 530 can optionally include one or more oscillators, mixers, frequency injectors, etc. to modulate and/or demodulate information on a carrier frequency to be transmitted and/or received by an antenna. In some examples, the device 500 is configured to indicate an output from the controller 540 by transmitting an electromagnetic or other wireless signal according to one or more wireless communications standards (e.g., Bluetooth, WiFi, IRdA, ZigBee, WiMAX, LTE) using communication system(s) 530. In some examples, the communication system(s) 530 could include one or more wired communications interfaces and the device 500 could be configured to indicate an output from the controller 540 by operating the one or more wired communications interfaces according to one or more wired communications standards (e.g., USB, FireWire, Ethernet, RS-232).

### Using Photoplethysmograph Waveform Data to Detect Cardiac Conditions Such as Atrial Fibrillation

Atrial fibrillation (AF) is a condition of the heart where the atria quiver and contract irregularly. Between 4 to 7 million people have AF in the United States; many are over 65 and hypertensive, or have other cardiovascular comorbidities. AF is the most common arrhythmia in adult populations. The prevalence is expected to continue to increase, reaching 12 million in the U.S. by year 2050. In addition to being widespread, AF contributes a significant cost burden to the healthcare system. Total AF-related direct spend in the U.S. is estimated to be 6.6 billion annually. However, AF detection is particularly sensitive to problems discussed above, in that existing metrics or features extracted from PPG data are not particularly reliable for detecting AF, and so it is difficult to obtain both adequate battery life and high-quality AF diagnostic information using a wearable device.

Currently in the marketplace, there is a lack of a noninvasive, long-term (i.e., >30 days) device for atrial fibrillation (AF) that incorporates clinically accepted signals (i.e., electrocardiogram). The absence of such a device has limited the ability of healthcare practitioners to know exactly if, when, and how much AF is occurring over the course of the patient's journey with his/her AF condition, thereby missing opportunities to deliver appropriate therapies in a timely manner or provide a feedback loop for existing rate/rhythm control strategies. “Silent”, or asymptomatic, AF is also a problem leading to undiagnosed and untreated AF which is associated with a 5x increased risk of stroke. Current screening methods and tools provide snapshot checks for AF, but are associated with a very low diagnostic yield due to the non-continuous data, which misses intermittent and unpredictable episodes of AF. Implantable devices such as pacemakers/ICDs and implantable loop recorders (ILRs) can technically provide this continuous data stream, but the high cost and invasive nature limit the market access and use case justification. A remote, low-cost, noninvasive technique to reliably monitor and detect AF on a long-term basis can fill the critical data gap needed to inform actionable medical interventions ultimately leading to improved patient outcomes.

In some embodiments of the present disclosure, the PPG sensor of the wearable device 400 records PPG waveform data, and transmits the PPG waveform data to a analysis device 500. The analysis device extracts features from the PPG waveform data, and provides the features to the classifier model 572 in order to detect likely instances of AF. In addition to traditional features extracted (such as IBI data), some embodiments of the present disclosure also use features related to the characteristics of the waveforms of individual beats to improve the predictions generated by the classifier model 572. In response to determining likely instances of AF based on these features, the wearable device 400 may prompt the user to take action to address the cardiac condition. In some embodiments, the IMU sensor 413 of the wearable device 400 also records data that is transmitted to the analysis device 500 in order to detect and filter motion artifacts from the PPG data.

In some embodiments, the recording of PPG waveform data by the wearable device 400 and the subsequent processing of the PPG waveform data by the analysis device 500 allows the wearable device 400 to provide non-invasive, long-term, continual monitoring, while also providing reliable detection of cardiac conditions without requiring further interaction with the subject beyond the collection of the PPG waveform data.

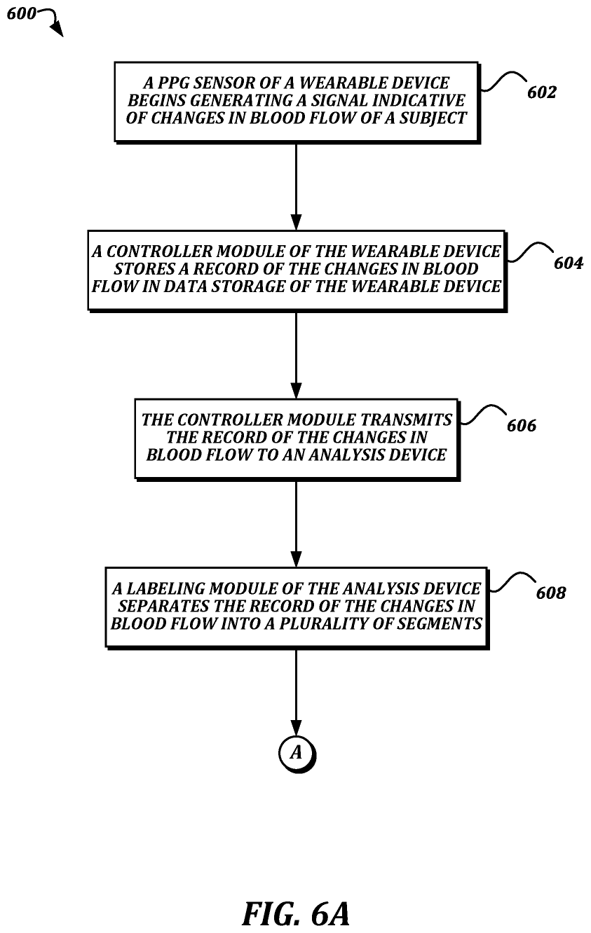

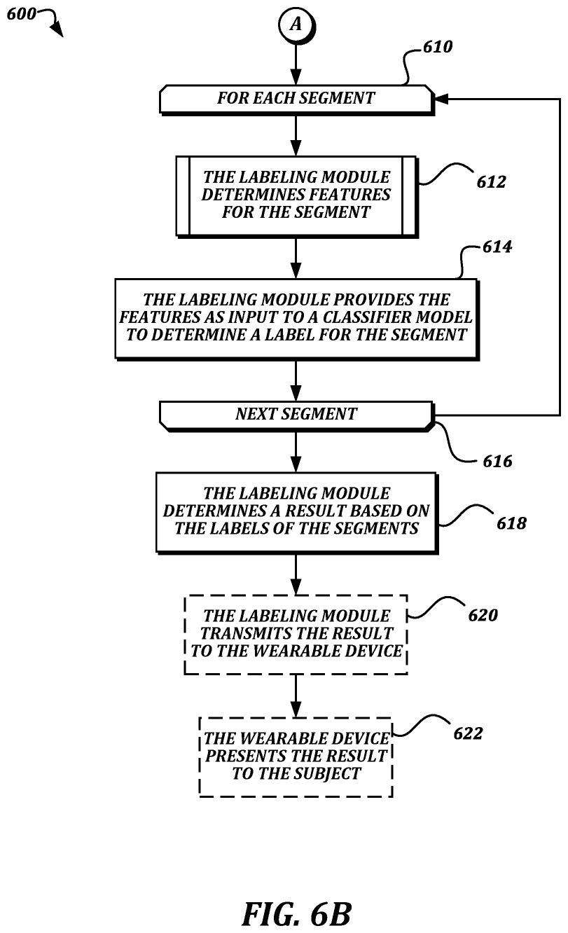

FIGS. 6A-6B are a flowchart that illustrates a non-limiting example embodiment of a method of monitoring cardiac activity to detect one or more conditions according to various aspects of the present disclosure. Atrial fibrillation (AF) is one cardiac condition for which the method 600 is particularly suited, but should not be seen as limiting. Any other cardiac condition that can be detected based on changes in blood flow that can be derived from the PPG waveform features described below may be monitored using a similar method.

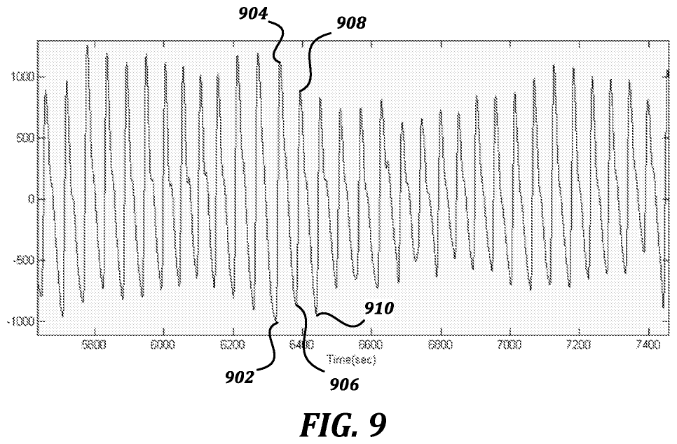

At block 602, a PPG sensor 412 of a wearable device 400 begins generating a signal indicative of changes in blood flow of a subject 100. In some embodiments, the signal may indicate an instantaneous level of blood flow at a given time, which may rise and fall throughout the cardiac cycle. In some embodiments, the signal will generate a new value at a given sampling rate, thus generating a sinusoidal waveform that represents the changes in blood flow during the cardiac cycle. Though a PPG sensor 412 is described, any other type of device that generates a waveform signal indicative of changes in blood flow of the subject 100 may be used. FIG. 9 is a chart that illustrates a non-limiting example embodiment of a signal generated by a PPG sensor 412 according to various aspects of the present disclosure, and will be discussed further below.

At block 604, a controller module 472 of the wearable device 400 stores a record of the changes in blood flow in data storage 460 of the wearable device 400. In some embodiments, the record of the changes in blood flow may be stored as a time series of values observed in the signal produced by the PPG sensor 412, or in any other suitable format. In some embodiments, the record may also include motion data generated by the IMU sensor 413.

At block 606, the controller module 472 transmits the record of the changes in blood flow (which may include the motion data) to a analysis device 500. In some embodiments, the controller module 472 may periodically (e.g., every 30 minutes, every hour, once a day, upon filling the data storage 460 to a predetermined percentage, etc.) transmit the record of the changes in blood flow to the analysis device 500 via a wireless communication technique. In some embodiments, the controller module 472 may transmit the record of the changes in blood flow via a wired communication technique, such as via USB, upon coupling of a wired communication system 430 of the wearable device 400 to a analysis device 500, or to an intermediary device that in turn transmits the record to the analysis device 500 via a high-bandwidth network connection. In some embodiments, a removable portion of the data storage 460 (such as an SD card) may be removed from the wearable device 400 and be physically provided to the analysis device 500 or the aforementioned intermediary device.

At block 608, a labeling module 576 of the analysis device 500 separates the record of the changes in blood flow into a plurality of segments. In some embodiments, the segments may be a length of time that allows multiple heartbeats to be depicted, but that are not too long such that short instances of a cardiac condition may be obscured. In some embodiments, about thirty seconds is a suitable length for a segment, though this length is an example only and should not be seen as limiting. In some embodiments, the segments may be split to each include a matching number of heartbeats, instead of to include a matching amount of time.

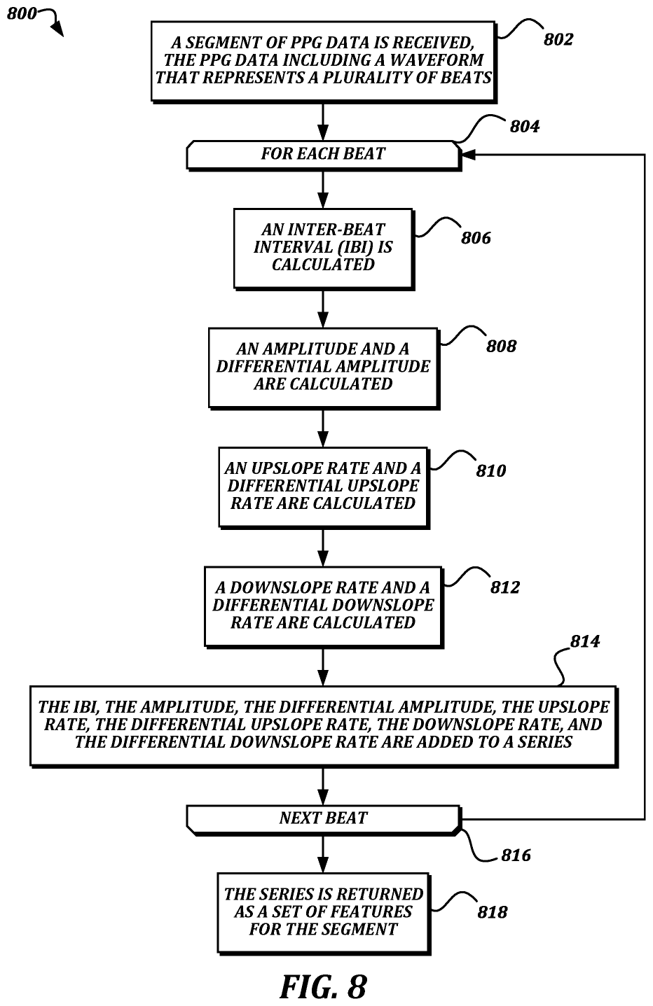

The method 600 then proceeds to a continuation terminal (“terminal A”). From terminal A (FIG. 6B), the method 600 proceeds to a for-loop defined between a for-loop start block 610 and a for-loop end block 616 wherein each segment of the plurality of segments is processed. From the for-loop start block 610, the method 600 advances to procedure block 612, where a procedure is executed wherein the labeling module 576 determines features for the segment. Any suitable procedure for determining features for the segment may be used. One example embodiment of such a procedure is illustrated in FIG. 8 and described in detail below.

At block 614, the labeling module 576 provides the features as input to a classifier model 572 to determine a label for the segment. The label may indicate the presence of a cardiovascular condition such as AF, the absence of a cardiovascular condition such as AF, or that the data is indeterminate. In some embodiments, more than one classifier model 572 may be used. For example, a first classifier model 572 may indicate a degree of likelihood that AF is present, and a second classifier model 572 may indicate a degree of likelihood that the segment is from a healthy subject. As another example, more classifier models 572 that have been trained to detect other cardiovascular conditions may also be used, such that more than one cardiovascular condition may be monitored. In some embodiments, instead of generating features in procedure block 612, the segment of the record of changes in blood flow may be provided directly to the classifier model 572 for processing and label generation.

The method 600 then advances to the for-loop end block 616. If further segments remain to be processed, then the method 600 returns to the for-loop start block 610 to process the next segment. Otherwise, the method 600 advances to block 618.

At block 618, the labeling module 576 determines a result based on the labels of the segments. In some embodiments, a result may indicate either a presence of the cardiovascular condition, an absence of the cardiovascular condition, or an indeterminate result. In some embodiments, a threshold number of segments (e.g., 20%, 30%) indicating that the cardiovascular condition is present may cause a result to be determined that indicates that the cardiovascular condition is present. In some embodiments, any single segment that indicates that the cardiovascular condition is present may cause a determination that the cardiovascular condition is present. In some embodiments, the result may reflect a percentage of segments in which the cardiovascular condition is detected. For example, the result may be provided as an AF burden value, which may be calculated based on the percentage of segments in which AF was detected and represents the proportion of time during which the subject is experiencing AF, an amount of time per day during which the subject is experiencing AF, a length of the longest episode of AF, or any other suitable measure.

At optional block 620, the labeling module 576 transmits the result to the wearable device 400, and at optional block 622, the wearable device 400 presents the result to the subject 100. In some embodiments, a value representing the presence or absence of the cardiovascular condition may be presented to the subject 100. In some embodiments, an indication of the urgency of the result may also be presented (e.g., healthy, vs. make an appointment with your doctor, vs. seek medical care immediately). Blocks 620 and 622 are illustrated as optional because, in some embodiments, the labeling module 576 may not transmit the result to the wearable device 400, and/or the wearable device 400 may not present the result to the subject.

Though the method 600 describes the wearable device 400 as transmitting the record to the analysis device 500 for processing, in some embodiments, the wearable device 400 may provide the classifier model 572 and the labeling module 576, or portions thereof, and may process the records locally. In some embodiments, portions of the processing of the data, such as the determination of features, may be performed on the wearable device 400, and then the determined features may be transmitted to the analysis device 500 for application of the classifier model 572.

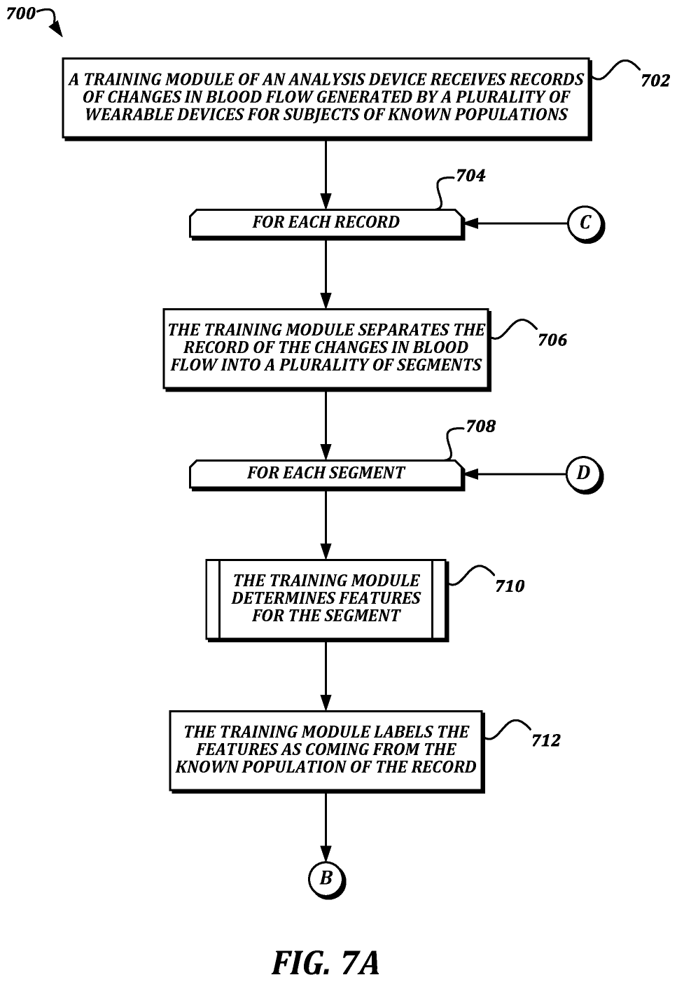

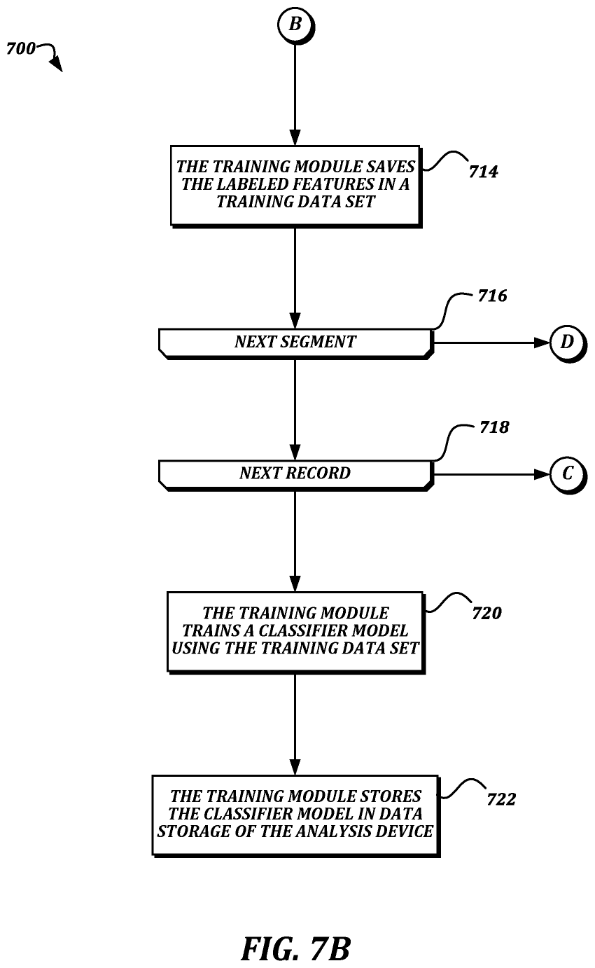

FIGS. 7A-7B are a flowchart that illustrates a non-limiting example embodiment of a method of training a classifier model to detect a cardiac condition according to various aspects of the present disclosure. In some embodiments, the method 700 is suitable for training a classifier model 572 as used in the method 600 described above.

At block 702, a training module 574 of a analysis device 500 receives records of changes in blood flow generated by a plurality of wearable devices 400 for subjects of known populations. In some embodiments, the records are recorded using a wearable device 400 that is similar (e.g., uses a similar type/model of PPG sensor 412 in a similar configuration) to the wearable device 400 to be used in the method 600 in which the trained classifier model 572 will be used. In some embodiments, at least some of the records may be generated using a different sensor or wearable device. In some embodiments, the records may be received directly from the wearable devices 400 upon the recording of the records. In some embodiments, the records may be obtained by the training module 574 from an archival source. Though a PPG sensor 412 is described, any other type of device that generates a waveform signal indicative of changes in blood flow of subjects may be used.

In some embodiments, the known populations include at least a population that is known to exhibit the cardiovascular condition and a population that is known to not exhibit the cardiovascular condition (e.g., a population that is known to be healthy). In some embodiments, the known populations may include further information about the subjects, including but not limited to sex, age, activity level, body mass index, or other factors that could help create multiple classifier models 572 that could be trained to more accurately detect cardiovascular conditions in sub-populations. In some embodiments, the information may also include motion information to help train the classifier model 572 to obtain accurate results in the presence of motion artifacts.

The method 700 then advances to a for-loop defined between a for-loop start block 704 and a for-loop end block 718, wherein each record of the records of changes in blood flow is processed. From the for-loop start block 704, the method 700 advances to block 706, where the training module 574 separates the record of the changes in blood flow into a plurality of segments. In some embodiments, the length of the segments matches the length of the segments into which the labeling module 576 creates at block 608, so that the training data and the test data remain similar. As such, the length of the segments may be a fixed length of time, a fixed number of heartbeats, or any other suitable length that matches the length used at block 608.

The method 700 then proceeds to another for-loop defined between a for-loop start block 708 and a for-loop end block 716, wherein each segment of the plurality of segments is processed. From the for-loop start block 708, the method 700 advances to procedure block 710, where a procedure is executed wherein the training module 574 determines features for the segment. Again, any suitable procedure for generating the features may be used, such as procedure 800 illustrated and described below, as long as the procedure in procedure block 710 matches the procedure by the labeling module 576 to generate features at procedure block 612.

At block 712, the training module 574 labels the features as coming from the known population of the record. In some embodiments, the labels may be an entry in a record, a row, a table, or another data structure that associates a value for the label with the record. The value for the label may indicate the characteristic of the known population with respect to the cardiovascular condition. For example, the label may indicate the presence or absence of the cardiovascular condition within the known population. In some embodiments, a separate label may not be applied to the features or segments themselves, but instead the features may be added to a set that is associated with the known population. The method 700 then proceeds to a continuation terminal (“terminal B”). From terminal B (FIG. 7B), the method 700 advances to block 714, where the training module 574 saves the labeled features in a training data set. In some embodiments, the training data set may be stored in the data storage 560. In some embodiments, motion data associated with the segment, and/or features extracted therefrom, may be added to the training data set as well. In some embodiments, instead of generating, storing, and labeling features to be added to the training data set, the segment of the record of changes in blood flow may be stored directly in the training data set and labeled, such that the classifier model 572 will be trained directly on the records of changes in blood flow.

The method 700 then advances to the for-loop end block 716. If further segments remain to be processed, then the method 700 returns from the for-loop end block 716 to the for-loop start block 708 via terminal D to process the next segment. Otherwise, the method 700 advances to the for-loop end block 718. If further records remain to be processed, then the method 700 returns from the for-loop end block 718 to the for-loop start block 704 via terminal C to process the next record. Otherwise, the method 700 advances to block 720.

At block 720, the training module 574 trains a classifier model 572 using the training data set. In some embodiments, each segment worth of features may be provided as training input, and the associated label may be provided as an expected output for the training input. As discussed above, any suitable type of classifier model 572 may be used, including but not limited to a support vector machine, a neural network, a decision tree, a naïve Bayes classifier, and a nearest neighbor model. Further, any suitable training technique may be used, including but not limited to gradient descent.

At block 722, the training module 574 stores the classifier model 572 in data storage 560 of the analysis device 500. In some embodiments, the training module 574 may also transmit the classifier model 572 to other devices that may use the classifier model 572, such as other analysis devices 500, or wearable devices 400 that have a labeling module 576 thereon.

FIG. 8 is a flowchart that illustrates a non-limiting example embodiment of a procedure for determining features for a segment of photoplethysmograph (PPG) data according to various aspects of the present disclosure. The procedure 800 is a non-limiting example of a procedure suitable for use at procedure block 612 and procedure block 710 in the above-described methods 600, 700. Though PPG data is described, in some embodiments, the waveform data that represents blood flow may be generated by a type of sensor device other than a PPG sensor.

At block 802, a segment of PPG data is received, the PPG data including a waveform that represents a plurality of beats. As discussed above, the waveform may be provided in any suitable format, including but not limited to a time series of blood flow values. In some embodiments, the PPG data may be pre-processed to remove motion and other artifacts. In some embodiments, information from an inertial measurement unit (IMU) of the wearable device 400 may be used to detect motion artifacts in the waveform. In some embodiments, additional features may be generated from the IMU data, and may be included in the sets of training data while training the classifier instead of pre-processing the PPG data using the IMU data. FIG. 9 is a chart that illustrates a non-limiting example embodiment of a PPG waveform according to various aspects of the present disclosure. The chart of FIG. 9 is not necessarily to scale, and may illustrate a waveform that is more or less regular than would be observed from a typical wearable device 400, but is suitable for discussion purposes herein. The horizontal axis illustrates the passage of time, while the vertical axis illustrates an amount of blood flow detected by the PPG sensor 412.

Returning to FIG. 8, the procedure 800 advances to a for-loop defined between a for-loop start block 804 and a for-loop end block 816, wherein each beat recorded in the segment of PPG data is processed. In some embodiments, the training module 574 may detect beats by searching for local maxima and minima, by analyzing the first derivative of the waveform to find inflection points, or using any other suitable method. The training module 574 may mark the beginning and end of each beat, such as by marking an entry in the time series associated with each determined peak, and marking an entry in the time series associated with each determined trough, with the waveform from a first trough, a first peak, and a second trough indicating a beat. In FIG. 9, trough 902 indicates the start of a first beat, peak 904 indicates the middle of the first beat, and trough 906 indicates the end of the first beat/start of a second beat. The second beat is defined from trough 906, peak 908, and trough 910. The remainder of the peaks and troughs in FIG. 9 are not labeled for the sake of clarity.

From the for-loop start block 804, the procedure 800 advances to block 806, where an inter-beat interval (IBI) is calculated. In some embodiments, the IBI may be the amount of elapsed time from a feature in one beat to a matching feature in the next beat. For example, in FIG. 9, the IBI between the first beat and the second beat may be measured by determining an amount of elapsed time from peak 904 to peak 908, or from trough 902 to trough 906.

At block 808, an amplitude and a differential amplitude are calculated. In some embodiments, the amplitude may be determined by computing the distance between the bottom of the waveform and the top of the waveform. In FIG. 9, the amplitude of the first beat may be determined based on a vertical difference between the peak 904 and the trough 902, by a vertical distance between the peak 904, and the trough 906, or by any other suitable measure for measuring waveform amplitude. In some embodiments, the differential amplitude may be determined by comparing an amplitude of the beat to an amplitude of a previous beat. In FIG. 9, the differential amplitude of the second beat (between trough 906 and trough 910) would be determined by comparing the amplitude of the second beat to the amplitude of the first beat (between trough 902 and trough 906).

At block 810, an upslope rate and a differential upslope rate are calculated. In some embodiments, the upslope rate may be determined based on an amount of time between a trough that indicates the start of a beat and a peak that indicates the middle of the beat, along with the difference in height between the trough that indicates the start of the beat and the peak. For example, in FIG. 9, the upslope rate of the first beat would be determined based on an amount of time between the trough 902 and the peak 904, as well as the vertical distance between the trough 902 and the peak 904. In some embodiments, the differential upslope rate may be determined by comparing the upslope rate of a beat to an upslope rate of a previous beat. In FIG. 9, the differential upslope rate of the second beat (the upslope rate between trough 906 and peak 908) would be determined by comparing that upslope rate to the upslope rate of the first beat (the upslope rate between trough 902 and peak 904).

At block 812, a downslope rate and a differential downslope rate are calculated. In some embodiments, the downslope rate may be determined based on an amount of time between a peak of a beat and a trough that indicates the end of the beat, along with the difference in height between the peak and the trough that indicates the end of the beat. For example, in FIG. 9, the downslope rate of the first beat would be determined based on an amount of time between the peak 904 and the trough 906, as well as the vertical distance between the peak 904 and the trough 906. In some embodiments, the differential downslope rate may be determined by comparing the downslope rate of a beat to a downslope rate of a previous beat. In FIG. 9, the differential downslope rate of the second beat (the downslope rate between peak 908 and trough 910) would be determined by comparing that downslope rate to the downslope rate of the first beat (the downslope rate between peak 904 and trough 906).

At block 814, the inter-beat interval (IBI), the amplitude, the differential amplitude, the upslope rate, the differential upslope rate, the downslope rate, and the differential downslope rate are added to a series. In some embodiments, the values may be combined in a data structure, and the data structure may be added to the series. In some embodiments, separate series may be created for each type of value, and the values may be added to each series separately.

The procedure 800 then advances to the for-loop end block 816. If further beats remain to be processed within the plurality of beats represented in the PPG data waveform, then the procedure 800 returns to the for-loop start block 804 to process the next beat. Otherwise, the procedure 800 advances to block 818, where the series is returned as a set of features for the segment.

Though a particular set of features was described in the procedure 800, in some embodiments, more, fewer, or different features may be used. For example, in some embodiments, IBI may be ignored. As another example, additional features may be determined based on the values that were discussed above, such as the average, the standard deviation, the kurtosis, or other statistical descriptions of the values discussed above.

While illustrative embodiments have been illustrated and described, it will be appreciated that various changes can be made therein without departing from the spirit and scope of the invention.

1. A system for monitoring a cardiac condition of a subject, the system comprising:
a wearable device that includes a photoplethysmograph (PPG) sensor; and
a computing system that is communicatively coupled to the wearable device and that includes logic that, in response to execution by the computing system, causes the computing system to perform actions comprising:
receiving PPG waveform data from the wearable device; and
providing at least information based on the PPG waveform data to a classifier model to label the PPG waveform data as either including the cardiac condition or not including the cardiac condition.

2. The system of claim 1, wherein providing at least information based on the PPG waveform data to the classifier model includes providing the PPG waveform data received from the wearable device to the classifier model.

3. The system of claim 1, wherein providing at least information based on the PPG waveform data to the classifier model includes:
generating features based on the PPG waveform data; and
providing the features to the classifier model.

4. The system of claim 3, wherein generating features based on the PPG waveform data includes determining, for each peak of the PPG waveform data, at least two of an inter-beat interval, an amplitude, an upslope rate, a downslope rate, a differential amplitude, a differential upslope rate, and a differential downslope rate.

5. The system of claim 1, wherein providing at least information based on the PPG waveform data includes:
separating the PPG waveform data into a plurality of segments; and
separately providing at least information based on the PPG waveform data associated with each segment of the plurality of segments to the classifier model to separately label each segment.

6. The system of claim 5, wherein the actions further comprise:
determining a percentage of segments that are labeled as including the cardiac condition; and
transmitting the percentage for presentation to the subject.

7. The system of claim 1, further comprising a hub device, wherein the wearable device is communicatively coupled to the computing system via the hub device.

8. The system of claim 1, wherein the wearable device further includes an inertial measurement unit (IMU) sensor; and wherein the actions further comprise:
receiving IMU data from the wearable device for a time period that coincides with the PPG waveform data; and
providing information based on the IMU data to the classifier model along with the features based on the PPG waveform data.

9. A non-transitory computer-readable medium having computer-executable instructions stored thereon that, in response to execution by one or more processors of a computing system, cause the computing system to perform actions for monitoring a subject for a cardiac condition, the actions comprising:
obtaining, by the computing system, photoplethysmograph (PPG) waveform data collected by a wearable device;
generating, by the computing system, features based on the PPG waveform data; and
providing, by the computing system, the features to a classifier model to label the PPG waveform data as either including the cardiac condition or not including the cardiac condition.

10. The computer-readable medium of claim 9, wherein generating features based on the PPG waveform data includes determining, for each peak of the PPG waveform data, at least two of an inter-beat interval, an amplitude, an upslope rate, a downslope rate, a differential amplitude, a differential upslope rate, and a differential downslope rate.

11. The computer-readable medium of claim 9, wherein generating features based on the PPG waveform data includes separating the PPG waveform data into a plurality of segments; and
wherein providing the features to the classifier model to label the PPG waveform data as either including the cardiac condition or not including the cardiac condition includes separately providing features associated with each segment of the plurality of segments to the classifier model to separately label each segment.

12. The computer-readable medium of claim 11, wherein the actions further comprise:
determining a percentage of segments that are labeled as including the cardiac condition; and
transmitting the percentage for presentation to the subject.

13. The computer-readable medium of claim 9, wherein the actions further comprise:
receiving inertial measurement unit (IMU) data from the wearable device for a time period that coincides with the PPG waveform data;
generating movement features based on the IMU data; and
providing the movement features to the classifier model along with the features based on the PPG waveform data.

14. The computer-readable medium of claim 9, wherein the computing system is separate from the wearable device, and wherein obtaining the PPG waveform data collected by the wearable device includes receiving the PPG waveform data via a network.

15. The computer-readable medium of claim 9, wherein the computing system is the wearable device, and wherein obtaining the PPG waveform data collected by the wearable device includes at least one of receiving the PPG waveform data from a PPG sensor of the wearable device and loading the PPG waveform data from a computer-readable medium of the wearable device.

16. A non-transitory computer-readable medium having computer-executable instructions stored thereon that, in response to execution by one or more processors of a computing system, cause the computing system to perform actions for training a classifier model to detect a cardiac condition, the actions comprising:
receiving, by the computing system, photoplethysmograph (PPG) waveform data collected from subjects of a first population that are known to exhibit the cardiac condition and from subjects of a second population that are known to not exhibit the cardiac condition;
generating, by the computing system, features based on the PPG waveform data to create a set of training data for the first population and a set of training data for the second population; and
training, by the computing system, at least one classifier model to label PPG waveform data as either exhibiting the cardiac condition or not exhibiting the cardiac condition using the set of training data for the first population and the set of training data for the second population.

17. The computer-readable medium of claim 16, wherein generating features based on the PPG waveform data includes determining, for each peak of the PPG waveform data, one or more of an amplitude, an upslope rate, a downslope rate, a differential amplitude, a differential upslope rate, and a differential downslope rate.

18. The computer-readable medium of claim 16, wherein generating features based on the PPG waveform data includes separating the PPG waveform data into a plurality of segments; and
wherein training the classifier model using the set of training data for the first population and the set of training data for the second population includes separately providing features for each segment of the plurality of segments as a separate training example.

19. The computer-readable medium of claim 16, wherein the actions further comprise:
receiving inertial measurement unit (IMU) data associated with the PPG waveform data;
generating movement features based on the IMU data; and
adding the movement features to the set of training data for the first population and to the set of training data for the second population.

20. The computer-readable medium of claim 16, wherein receiving PPG waveform data collected from subjects of a first population that is known to exhibit the cardiac condition and from subjects of a second population that is known to not exhibit the cardiac condition includes receiving PPG waveform data from a plurality of wearable devices.
